(19) United States
(12) Patent Application Publication
(10) Pub. No.: US 2003/0181461 A1
(43) Pub. Date: Sep. 25, 2003

(54) Use of phosphodiesterase antagonists to treat insulin resistance

(76) Inventors: Wilfred Wayne Lautt, Winnipeg (CA); Paula Macedo, Caxias (PT)

Correspondence Address: Merchant & Gould P.C., P.O. Box 2903, Minneaposis, MN, 55402-0903, US

(21) Appl. No.: 10/350,070

(22) Filed: Jan. 24, 2003

Related U.S. Application Data
(60) Provisional application No. 60/350,954, filed on Jan. 25, 2002.

(51) Int. Cl. A61K 31/519; A61K 31/522; A61K 9/127; A61K 38/38

(52) U.S. Cl. 514/252.16; 424/450; 514/263.34; 514/262.1; 514/2

(57) ABSTRACT

A phosphodiesterase antagonist is used to reduce insulin resistance, and to amplify the effect of nitric oxide on skeletal muscle insulin-mediated glucose uptake in a mammal. In some instances, the antagonist is targeted to the liver. In some instances, the insulin resistance is hepatic insulin sensitizing substance (“HISS”) dependant insulin resistance.

This application claims priority of invention from U.S. patent application Ser. No. 60/350,954, filed Jan. 25, 2002.

### FIELD OF THE INVENTION

The invention relates to the field of treatments for insulin resistance.

### BACKGROUND

Insulin resistance is a significant health challenge for a wide range of patients, including those with type II diabetes, metabolic obesity, and various liver conditions.

The picture that is emerging is one of complex multiple interacting systems with reflex parasympathetic effects in the liver capable of causing more than one reaction and of triggering reactions in other organs.

In fasted cats, hypoglycemic response to a bolus administration of insulin was reduced by 37% by hepatic denervation. These cats developed insulin resistance immediately following acute denervation of the liver. The degree of reduction of response to insulin was maximal after anterior plexus denervation and did not increase further with addition of denervation of the posterior nerve plexus or bilateral vagotomy thus demonstrating that all of the nerves of relevance were in the anterior plexus. To avoid the complexity of the reaction to hypoglycemia, the rapid insulin sensitivity test (RIST) was employed (Lautt et al., Can. J. Physiol. Pharmacol. 76:1080 (1998)) wherein a euglycemic clamp was used following the administration of insulin and the response was quantitated as the amount of glucose required to be infused over the test period in order to hold arterial blood glucose levels constant. The RIST methodology has been published in detail and has been demonstrated in both cats and rats. It is highly reproducible. Insulin, glucagon, and catecholamine levels remain unchanged between tests.

Cats showed a dose-related development of insulin resistance using atropine (a cholinergic muscarinic receptor antagonist) that was of a similar magnitude to that produced by surgical denervation. The dose of atropine required to produce a full insulin resistance is 3 mg/kg (4 μmol/kg) administered into the portal vein. A similar degree of insulin resistance was achieved with 10⁻⁷ mmol/kg of the M₁ muscarinic selective antagonist, pirenzepine, and with 10⁻⁶ μmol/kg of the M₂ selective antagonist, methoctramine. Although not conclusive, the data suggest that the response may be mediated by the M₁ muscarinic receptor subtype.

Although the liver appeared to be the organ that produced the insulin resistance, it was not clear that the liver was the resistant organ. In order to determine the site of insulin resistance, a further series was done in cats that measured arterial-venous glucose responses across the hindlimbs, extrahepatic splanchnic organs, and liver. The intestine was unresponsive to the bolus insulin administration both before and after atropine or anterior plexus denervation or the combination of both. The hepatic response was also not notably altered whereas the glucose uptake across the hindlimbs, primarily representing skeletal muscle uptake, was decreased following atropine or hepatic parasympathetic denervation. These results indicated that interference with hepatic parasympathetic nerves led to insulin resistance in skeletal muscle.

It was further demonstrated that the same degree of resistance could be produced by pharmacological blockade of parasympathetic nerve function using the muscarinic receptor antagonist, atropine. Following a meal, insulin is released from the pancreas. The presence of insulin in the blood elicits a hepatic parasympathetic reflex that results in the release of acetylcholine in the liver that results in the generation and release of nitric oxide which acts to control the sensitivity of skeletal muscle to insulin through the action of a hormone released from the liver, a hepatic insulin sensitizing substance (HISS) which selectively stimulates glucose uptake and storage as glycogen in tissues including skeletal muscle.

In the absence of HISS, the large muscle mass is highly resistant to insulin and the glucose storage in skeletal muscle is severely reduced. Interruption of any part of the parasympathetic-mediated release of HISS results in insulin resistance. This parasympathetic reflex regulation of HISS release is a fundamental mechanism by which the body regulates responsiveness to insulin and this mechanism is adjusted according to the prandial state, that is, according to how recently there has been a consumption of nutrients.

In a fasted condition, HISS release in response to insulin is minimal or absent so that if insulin is released in this situation, there is a minimal metabolic effect. Following a meal, the parasympathetic reflex mechanism is amplified so that HISS release occurs and results in the majority of the ingested glucose stored in skeletal muscle.

The consequence of lack of HISS release is the absence of HISS which results in severe insulin resistance, referred to as HISS-dependent insulin resistance (“HDIR”). In this situation, the pancreas is required to secrete substantially larger amounts of insulin in order that the glucose in the blood is disposed of to prevent hyperglycemia from occurring. If this condition persists, insulin resistance will progress to a state of type 2 diabetes (non-insulin dependent diabetes mellitus) and eventually will lead to a complete exhaustion of the pancreas thus requiring the patient to resort to injections of insulin. Thus, it appears that any condition in which the hepatic parasympathetic reflex is dysfunctional will result in insulin resistance.

It is believed that the insulin resistance that is seen: in a variety of conditions (non-insulin dependent diabetes, essential hypertension, obesity, chronic liver disease, fetal alcohol effects, old age, and chronic inflammatory diseases) represents a state of HDIR parasympathetic dysfunction. Lack of HISS would also be anticipated to result in obesity at the early stage of the resultant metabolic disturbance (the obese often become diabetic).

Normally after a meal, the liver takes up a small proportion of glucose and releases HISS to stimulate skeletal muscle to take up the majority of the glucose load. In the absence of HISS, the skeletal muscle is unable to take up the majority of glucose thus leaving the liver to compensate. The hepatic glycogen storage capacity is insufficient to handle all of the glucose, with the excess being converted to lipids which are then incorporated into lipoproteins and transported to adipose tissue for storage as fat. Provision of HISS to these individuals would restore the nutrition partitioning so that the nutrients are stored primarily as glycogen in the skeletal muscle rather than as fat in the adipose tissue.

Thus, it is an object of the invention to provide a method of reducing insulin resistance.

### SUMMARY OF THE INVENTION

The invention provides uses and methods for reducing insulin resistance. Insulin resistance of certain tissues, including skeletal muscle, is modulated by the splanchnic reflex, which is normally triggered by consumption of a meal. Downstream of the splanchnic reflex, changes necessary for the reduction of insulin resistance are triggered by increased levels of cyclic GMP (“cGMP”). Thus, in one embodiment the invention provides a method of reducing insulin resistance by inhibiting the breakdown of cGMP.

In another embodiment of the invention there is provided the use of a phosphodiesterase antagonist to reduce insulin resistance in a patient suffering therefrom.

### BRIEF DESCRIPTION OF THE FIGURES

FIG. 1 is a graphical depiction of the effect of zaprinast on insulin sensitivity in atropine-treated rats.

### DETAILED DESCRIPTION OF THE PREFERRED EMBODIMENTS

Insulin resistance is modulated by a multi-step process normally initiated by the consumption of a meal. The consumption of a meal results in the release of acetylcholine, which is believed to activate muscarinic receptors, leading ultimately to an increase in guanyl cyclase activity and an increase in the level of cGMP. This normal pathway can be blocked by in number of disease states, as well as by hepatic denervation. Such blockage can be mimiced by the administration of atropine, which blocks normal acetylcholine release, reducing or preventing normal activation of the hepatic muscarinic receptors. Such blockage interferes with the release of hepatic insulin sensitizing substance (“HISS”) which is necessary for normal insulin sensitivity in some tissues, including skeletal muscle.

The present invention provides methods and uses alleviating the symptoms of such blockages by increasing the effective level of cGMP available to stimulate a reduction in HISS-dependent insulin resistance (“HDIR”). HDIR is a reduction in the response to insulin secondary to a failure of HISS action on glucose disposal. When insulin fails to result in HISS release from the liver or its action on skeletal muscle is otherwise impaired, a state of HDIR is said to exist. In a pure state of HDIR, the direct glucose uptake stimulation effect of insulin is not impaired.

Cats show a dose-related development of insulin resistance using atropine that was of a similar magnitude to that produced by surgical denervation. The dose of atropine required to produce a full insulin resistance was 3 mg/kg (4 μmol/kg) administered into the portal vein. A similar degree of insulin resistance was achieved with 10⁻⁷ mmol/kg of the M₁ muscarinic selective antagonist, pirenzepine, and with 10⁻⁶ μmol/kg of the M₂ selective antagonist, methoctramine. The data suggest that the response may be mediated by the M₁ muscarinic receptor subtype.

In order to determine the site of insulin resistance, a further series was done in cats that measured arterial-venous glucose responses across the hindlimbs, extrahepatic splanchnic organs, and liver. The intestine was unresponsive to the bolus insulin administration both before and after atropine or anterior plexus denervation or the combination of both. The hepatic response was also not notably altered whereas the glucose uptake across the hindlimbs, primarily representing skeletal muscle uptake, was decreased following atropine or hepatic parasympathetic denervation. These results indicated that interference with hepatic parasympathetic nerves leads to insulin resistance in skeletal muscle.

It was further demonstrated that the same degree of resistance could be produced by pharmacological blockade of parasympathetic nerve function using the muscarinic receptor antagonist, atropine.

While the invention is not limited to any particular model or mechanism of action, it appears that in normal individuals, the eating of a meal results not only in the release of insulin, but also in a hepatic parasympathetic reflex. The hepatic parasympathetic effect results in the release of acetylcholine (ACh) which activates muscarinic receptors in the liver. This activation leads to increased guanyl cyclase activity, resulting in increased levels of cyclic guanosine monophosphate (cGMP) which increases activated intracellular hepatic glutathione which acts in stimulating the release into the blood of a hepatic insulin sensitizing substance (HISS) which leads to an increase in insulin sensitivity in skeletal muscle.

Thus, the invention provides, in one embodiment, a method of increasing glucose uptake by skeletal muscle of a patient suffering from suboptimal hepatic regulation of blood glucose levels by administering a suitable phosphodiesterase antagonist.

In some instances it will be desirable to enhance the response to cGMP. For example, where an injury or abnormality causes reduced cGMP production in response to a meal, it is desirable to amplify the effect of the cGMP which is produced. Similarly, where cGMP, ACh, or a similar compound involved in the normal response to a meal is absent or present only at insufficient levels, and cGMP (or another suitable compound acting prior to cGMP and leading to cGMP production in the liver) must be provided exogenously to reduce insulin resistance, such as through medication, it is desirable to enhance the effectiveness of the exogenously supplied compound. Also, where cGMP is produced at normal levels, but due to disease or other abnormality, activated glutathione is produced at lower than normal levels, it may be desirable to increase the effectiveness of the endogenous cGMP. This can be accomplished by reducing the rate at which cGMP is degraded by phosphodiesterases in the liver, thereby increasing the cGMP available and allowing the effect of a given cGMP molecule on insulin sensitivity to be amplified. Similarly, the effect of NO release on insulin sensitivity can be amplified by administering a cGMP phosphodiesterase antagonist.

During normal liver function, intracellular cGMP is broken down by its phosphodiesterase. This prevents the unlimited build-up of cGMP in liver cells, which, in normal patients, could result in an undesirably high level of activated glutathione production long after the initial release of NO which lead to cGMP synthesis.

Phosphodiesterase subtypes 3 and 5 are believed to be responsible for the breakdown of cGMP. Thus, in some instances it will be desirable to inhibit the function of phosphodiesterase subtype 3 and/or 5.

Non-limiting examples of antagonists of phosphodiesterase subtypes 3 and 5 are vinpocetine, zaprinast and dipyridamole, and sildenafil. Non-limiting examples of other phosphodiesterase antagonists which might be desirable to use in some situations are: theophylline, aminophylline, isobutylmethyl xanthine anagrelide, tadalafil, dyphylline, vardenafil, cilostazol and caffeine.

Where it is desired to antagonize phosphodiesterases of subclasses other than 3 and 5 the following compounds may be employed: milrinone, amrinone pimobendan, cilostamide, enoximone, teroximone, vesmarinone, rolipram and R020-1724.

In one embodiment of the invention, a cGMP phosphodiesterase (cGMP PE) antagonist, is used to reduce the breakdown of cGMP in liver cells. The precise dose and method of administration of the cGMP phosphodiesterase antagonist desirable will be determined by a number of factors which will be apparent to those skilled in the art, in light of the disclosure herein. In particular, the identity of the antagonist, the formulation and route of administration employed, the patient's gender, age and weight, as well as the extent of cGMP production in the hepatic parasympathetic neurons of interest, the number and effectiveness of the cGMP response in liver cells and the severity of the condition to be treated should be considered. Where it is impractical to conduct the tests necessary to determine the cGMP response and/or the other factors such as the extent of hepatic cGMP production, the appropriate dose can be determined through the administration of a dose suitable for a majority of patients similar to the subject in respect of those factors which have been assessed, followed by routine monitoring of insulin resistance (via RIST) where the dose provided does not cause insulin resistance to decline to normal or tolerable levels, the dose should be increased. The patient should be monitored for signs of excess cGMP PE antagonist exposure.

In some instances it will be desirable to administer the phosphodiesterase antagonist intravenously at a dose of between about 5 and 500 μg/kg body weight. In some instances a dose of between about 50 μg/kg and 150 μg/kg body weight will be desirable. In some cases an intravenous dose of 50 μg/kg to 70 μg/kg will be desirable.

In some instances it will be desirable to administer the phosphodiesterase antagonist orally at between about 1 to 500 mg/kg body weight. In some instances, an oral dose of between about 2 mg/kg and 300 mg/kg body weight will be desired. In some instances oral doses in the range of 10 to 100 mg/kg body weight will be desired. In some instances an oral dose of 15 to 50 mg/kg body weight will be desired. The phosphodiesterase antagonist will frequently be administered so as to ensure that it reaches maximum plasma concentrations just prior to the meal and remains high for at least one hour and preferably no more than 4 to 6 hours thereafter. For example, sildenafil (when administered orally) typically reaches maximum plasma concentrations within 30 to 120 minutes of administration. Thus, the oral dose (e.g. 50 mg) would be taken approximately 30 minutes prior to the meal.

In some instances, transdermal administration or intraperitoneal administration of the phosphodiesterase antagonist will be desired.

Any suitable phosphodiesterase antagonist may be employed. A phosphodiesterase antagonist will be “suitable” if: (a) at the dose and method of administration to the mammalian patient, it is not acutely toxic, and does not result in chronic toxicity disproportionate to the therapeutic benefit derived from treatment; and (b) at the dose and method of administration to the mammalian patient it reduces insulin resistance in the patient.

It will be apparent that a combination of phosphodiesterase antagonists (specific for the same or different subtypes, or non-specific) may be administered.

The phosphodiesterase antagonist may be administered together with one or more acetylcholine esterase antagonists as described in the co-pending International Patent Application claiming priority from U.S. Pat. No. 60/350,958, filed on Jan. 25, 2002 of Lautt.

The phosphodiesterase antagonist may be administered together with other drugs used in the treatment of diabetes, non-limiting examples of which are provided in Table 1. In some instances, a pharmaceutical composition comprising a suitable phosphodiesterase antagonist and one or more other drugs used in the treatment of diabetes will be desired.

TABLE I
a. Insulin and insulin analogues
b. Type II Diabetes drugs

| | |
|---|---|
| i. | Sulfonylurea agents |

1. First Generation

a. Tolbutamide
b. Acetohexamide
C. Tolazamide
d. Chlorpropamide

2. Second Generation

a. Glyburide
b. Glipizide
c. Glimepiride

| | |
|---|---|
| ii. | Biguanide agents |

1. metformin

| | |
|---|---|
| iii. | Alpha-glucosidase inhibitors |

1. Acarbose
2. Miglitol

| | |
|---|---|
| iv. | Thiazolidinedione Agents (insulin sensitizers) |

1. Rosiglitazone
2. Pioglitazone
3. Troglitazone

| | |
|---|---|
| v. | Meglitinide Agents |

1. Repaglinide

c. Cholinesterase Inhibitors

| | |
|---|---|
| i. | Donepezil |
| ii. | Tacrine |
| iii. | Edrophonium |
| iv. | Demecarium |
| v. | Pyridostigmine |
| vi. | Phospholine |
| vii. | Metrifonate |
| viii. | Neostigmine |
| ix. | Galanthamine |
| x. | Zanapezil |

d. Cholinergic Agonists

| | |
|---|---|
| i. | Acetylcholine |
| ii. | Methacholine |
| iii. | Bethanechol |
| iv. | Carbachol |
| v. | Pilocarpine hydrochloride |

e. Nitric Oxide Donors

| | |
|---|---|
| i. | Products or processes to increase NO synthesis in the liver |
| | (increasing NO synthase activity) |

Variety I

1. SIN-1
2. Molsidamine

Variety II

1. N-acetylcysteine
2. Cysteine esters
3. L-2-oxothiazolidine-4-carboxolate (OTC)
4. Gamma glutamylcystein and its ethyl ester
5. Glutathione ethyl ester
6. Glutathione isopropyl ester
7. Lipoic acid
8. Cysteine
9. Cystine
10. Methionine
11. S-adenosylmethionine

| | |
|---|---|
| ii. | Products or processes to reduce the rate of NO degradation |
| | in the liver |
| iii. | Products or processes to provide exogenous NO or an |
| | exogenous carrier or precursor which is taken up and |
| | releases NO in the liver |

f. Antioxidants

| | |
|---|---|
| i. | Vitamin E |
| ii. | Vitamin C |
| iii. | 3-morpholinosyndnonimine |

g. Glutathione increasing compounds

| | |
|---|---|
| i. | N-acetylcysteine |
| ii. | Cysteine esters |
| iii. | L-2-oxothiazolidine-4-carboxolate (OTC) |
| iv. | Gamma glutamylcystein and its ethyl ester |
| v. | Glutathione ethyl ester |
| vi. | Glutathione isopropyl ester |
| vii. | Lipoic acid |
| viii. | Cysteine |
| ix. | Cystine |
| x. | Methionine |
| xi. | S-adenosylmethionine |

In one embodiment, the phosphodiesterase antagonist is preferentially targeted to the liver. Targeting of the antagonist to the liver can be accomplished through the use of any pharmaceutically acceptable liver-targeting substance. For example, it can be bound to albumin or bile salts for preferential delivery to liver; alternatively, the antagonist may be incorporated into or encapsulated within liposomes which are preferentially targeted to the liver. In one embodiment, the antagonist is administered in a precursor form, and the precursor is selected to be metabolised to the active form by enzymes preferentially found in the liver.

In light of the disclosure herein, one skilled in the art could readily determine if a particular candidate cGMP PE antagonist is a suitable antagonist by determining the method and dose of administration and performing toxicity studies according to standard methods (generally beginning with studies of toxicity in animals, and then in humans if no significant animal toxicity is observed). If the method and dose of administration do not result in acute toxicity, the antagonist is administered to the subject at the dose and method of administration for at least 3 days. Insulin resistance following treatment for at least three days is compared to pre-treatment insulin resistance. (Insulin resistance is assessed using the RIST test). Where treatment results in decreased insulin resistance without significant chronic toxicity (or having only modest chronic toxicity in a patient where untreated insulin resistance is life threatening), the antagonist is a suitable antagonist for that patient at the dose and method tested.

Methods for conducting toxicity studies are known in the art. Of particular interest with respect to phosphodiesterase antagonist toxicity are tests of liver function and cardiovascular observations.

It will often be desirable to monitor patients receiving phosphodiesterase antagonists for signs suggesting excessive exposure to the antagonist, including decreased blood pressure, headache, flushing of the face, and nasal congestion. Erection may be observed as a side-effect in male patients but is not necessarily indicative of excess phosphodiesterase antagonist exposure.

The patient is preferably mammalian. In one embodiment the patient is a human being. In another embodiment the patient is a domestic animal such as a cat, dog, or horse. Where the patient is a ruminant animal, it may be particularly desirable to test blood glucose levels after a meal to determine the rate of blood glucose elevation and select a suitable time for antagonist administration. In some instances it may be desirable to screen a potential patient to confirm that he or she suffers from HDIR prior to administering a phosphodiesterase antagonist.

The phosphodiesterase antagonist may be administered so as to maintain a relatively constant level of the antagonist in the liver at all times. Alternatively, the antagonist may be administered to have antagonist concentrations peak when blood glucose is high, such as after a meal, so as to allow glucose uptake at that time. Where toxicity is a concern, it may be desirable to keep antagonist levels low until blood glucose levels become elevated above normal levels.

In one embodiment of the invention there is provided a method of reducing insulin resistance in a mammalian patient suffering from insufficient levels of hepatic cGMP. The method comprises: selecting a patient suffering from above average levels of insulin resistance, and administering a suitable cGMP phosphodiesterase antagonist.

As used herein, the phrase “insufficient levels of hepatic cGMP” means levels of hepatic cGMP insufficient to reduce insulin resistance to the average level observed in healthy subjects of the same gender, age, weight, fed-state, and blood glucose level as the patient.

As used herein, the phrase “above average levels of insulin resistance” means levels of insulin resistance above the average level observed in healthy subjects of the same gender, age, weight, fed-state, and blood glucose level as the patient.

In one embodiment of the invention there is provided a kit containing a phosphodiesterase antagonist in a pharmaceutically acceptable carrier together with instructions for the administration of the cGMP phosphodiesterase antagonist to reduce insulin resistance in a patient. In one embodiment the kit further includes means to administer the cGMP phosphodiesterase antagonist. Suitable means may be selected by one skilled in the art, depending on the route of administration desired.

Individuals suffering from insulin resistance who could in many cases benefit from treatment according to the methods described herein include those suffering from any one or more of: chronic liver disease, chronic hypertension, type II diabetes, fetal alcohol syndrome, gestational diabetes, obesity, and age-related insulin resistance as well as liver transplant recipients.

L-NAME and L-NMMA are nitric oxide synthase (NOS) antagonists. SIN-1 is a nitric oxide (NO) donor. Atropine interrupts the hepatic reflex response to insulin.

### EXAMPLE

Methods for reversal of HISS-dependent insulin resistance using a phosphodiesterase inhibitor in normal rats

Male Sprague Dawley rats (250-300 g) were allowed free access to water and normal rodent food for 1 week prior to all studies. Rats were fasted for 8 hours overnight and fed for 2 hours before the start of study.

Rats were anesthetized with pentobarbital-sodium (65 mg/ml, ip injection, 0.1 ml/100 g body weight). Animals were placed on a heated thermostatically controlled surgical table to maintain body temperature during surgery and the experimental procedure.

An extra-corporeal arterial-venous shunt (the loop) was established between the right femoral artery and right femoral vein, according to a published, standard operating procedure developed in our laboratory (Xie et al., 1996). The loop allows for regular blood sampling of arterial blood throughout the experiment as well as infusion of intravenous drugs and monitoring of arterial blood pressure.

A tracheal breathing tube was inserted to ensure a patent airway and the jugular vein was cannulated for administration of supplemental anesthetic through out the study, and 10% w/vol glucose solution during the insulin sensitivity test procedure (rapid insulin sensitivity test, RIST). A laparotomy was performed and an indwelling portal venous catheter was inserted using a portal vein puncture technique. The portal catheter was used to administer the phosphodiesterase inhibitor directly to the liver.

The Rapid Insulin Sensitivity Test (the RIST) is a euglycemic approach to test whole body glucose uptake in response to a low dose insulin challenge. It has been extensively validated against other standard approaches and has proven to be a sensitive, reliable and reproducible technique (Reid, et al., 2002).

Once surgery was completed, the rat was allowed to stabilize for approximately 30 minutes. At this point, blood samples (25 μl) were taken at regular intervals from the loop and analyzed for glucose concentration. Once a stable baseline glucose level was obtained, animals were given a 5 minute infusion of insulin (50 mU/kg) through the loop. Glucose levels were monitored every 2 minutes during and after the infusion of insulin. Exogenous glucose was infused into the jugular vein to prevent the hypoglycemic effect of insulin. Based on the glucose levels obtained from the regular blood sampling, the infusion rate of glucose was adjusted to maintain the baseline euglycemia. Glucose infusion rates progressively increased as the effect of insulin reached a maximum (at approximately 15 minutes into the test) and then progressively decreased as the effect of insulin wore off. Typically, the effect of insulin is complete by 35 minutes. The total amount of glucose infused during the RIST is considered the RIST index and is reported in terms of mg glucose infused/kg body weight of the subject.

As some degree of neural activation must remain for the phosphodiesterase inhibitors to be effective, an atropine model of 75% blockade of HISS-dependent insulin resistance (HDIR) was developed. The dose of atropine used (5×10⁻⁶ mg/kg) was based on previously obtained dose-response data obtained in the rat. To this end, atropine was infused into the loop for 5 minutes. After allowing time to re-establish a stable blood glucose level, a RIST was performed to determine the degree of insulin resistance.

Zaprinast is a phosphodiesterase inhibitor and prevents the metabolism of cyclic guanosine monophosphate (cGMP).

After determining the degree of insulin resistance produced by atropine, a 0.5 ml bolus of zaprinast was infused into the portal vein at a dose of 0.1 mg/kg over 20 minutes. A stable blood glucose baseline was established and a RIST was conducted to determine if this agent could reverse the insulin resistance.

Summary of experimental protocol

1. control RIST to determine insulin sensitivity

2. atropine infusion to produce a 75% block of HISS-dependent insulin resistance

3. post-atropine RIST

4. bolus infusion of zaprinast into portal vein

5. RIST after zaprinast administration

Human insulin (Humulin R) was obtained from Eli Lilly and Company. Atropine and zaprinast were obtained from Sigma Chemical Company. Insulin and atropine were diluted or dissolved in normal saline. Zaprinast was suspended in saline and then dissolved with 200 μl of 1 N sodium hydroxide (NaOH). The solution was then titrated to a pH of 8-9 using 1 M hydrochloric acid (HCl).

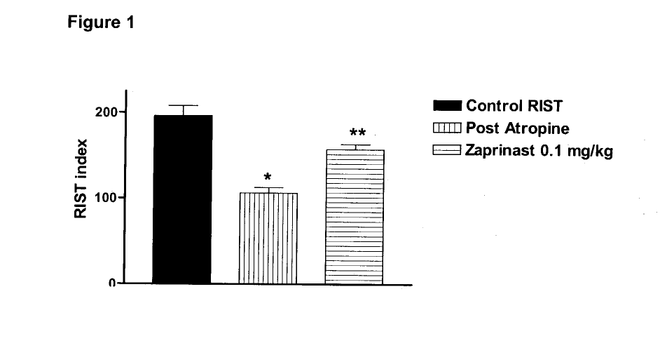

As shown in FIG. 1, the average control RIST index was 196.5±11.4 mg /kg (n=8). Following the atropine-induced 75% HDIR, the RIST index was significantly decreased to 106.1±6.3 mg/kg (*). The RIST index following the zaprinast (0.1 mg/kg, ipv) was increased to 157.4±6.1 mg/kg and was significantly increased from the blocked state (**). These data indicate that zaprinast is able to reverse the HDIR produced by atropine.

Thus, it will be apparent that there has been provided a method of reducing insulin resistance.

We claim:

1. A method of reducing insulin resistance in a mammalian patient comprising administering a suitable phosphodiesterase antagonist.

2. The method of claim 1 wherein the insulin resistance is HISS-dependent insulin resistance.

3. The method of claim 2 wherein the antagonist is at least one of vinpocetine, zaprinast and dipyridamole, sildenafil, theophylline, aminophylline, isobutylmethyl xanthine anagrelide, tadalafil, dyphylline, vardenafil, cilostazol and caffeine.

4. The method of claim 2 wherein the phosphodiesterase antagonist is antagonist of at least one of phosphodiesterase of subtype 3 and 5.

5. The method of claim 2 wherein the phosphodiesterase antagonist is preferentially targeted to the liver.

6. The method of claim 5 wherein the phosphodiesterase antagonist is targeted to the liver using albumin.

7. The method of claim 5 wherein the phosphodiesterase antagonist is targeted to the liver using a plurality of liposomes.

8. The method of claim 1 wherein the phosphodiesterase antagonist is administered by intravenous administration.

9. The method of claim 1 wherein the phosphodiesterase antagonist is administered by transdermal administration.

10. The method of claim 1 wherein the phosphodiesterase antagonist is administered by oral administration.

11. The method of claim 1 wherein the phosphodiesterase antagonist is administered by intra peritoneal administration.

12. The method of claim 1 wherein the phosphodiesterase antagonist is administered by portal vein injection.

13. The method of claim I wherein the phosphodiesterase antagonist is administered orally at a dose of between about 2 and 300 mg/kg body weight.

14. The method of claim 1 wherein the phosphodiesterase antagonist is administered intravenously at a dose of between about 5 and 500 μg/kg body weight.

15. The method of claim 1 wherein the patient is a human.

16. A method of amplifying the effect of nitric oxide on skeletal muscle insulin sensitivity comprising administering a phosphodiesterase antagonist.

17. The method of claim 16 wherein the phosphodiesterase antagonist is at least one of vinpocetine, zaprinast and dipyridamole, sildenafil, theophylline, aminophylline, isobutylmethyl xanthine anagrelide, tadalafil, dyphylline, vardenafil, cilostazol and caffeine.

18. A kit comprising:
a phosphodiesterase antagonist in a pharmaceutically acceptable carrier; and
instructions for the administration of the phosophodiesterase antagonist to reduce insulin resistance in a mammalian patient.

19. The kit of claim 18 further comprising means to administer the phosphodiesterase antagonist.

20. A pharmaceutical composition comprising a phosphodiesterase antagonist and at least one other drug used in the treatment of diabetes.

21. The pharmaceutical composition of claim 20 further including a pharmaceutically acceptable liver-targeting substance.

22. A method of increasing glucose uptake by skeletal muscle of a patient, comprising administering a phosphodiesterase antagonist.

23. The method of claim 22 wherein the phosphodiesterase antagonist is preferentially targeted to the liver.

24. The method of claim 22 wherein the phosphodiesterase antagonist includes at least one of vinpocetine, zaprinast, dipyridamole, sildenafil, theophylline, aminoptiylline, isobutylmethyl xanthine and caffeine.

25. The method of claim 22 wherein the patient suffers from at least one of chronic liver disease, chronic hypertension, type II diabetes, fetal alcohol syndrome, gestational diabetes, obesity, age-related insulin resistance, and hepatic nerve damage.

26. The method of claim 25 wherein the patient is a human.

27. The method of claim 24 wherein the phosphodiesterase antagonist is administered orally at a dose of between about 2 and 300 mg/kg body weight.

28. The method of claim 25 wherein the phosphodiesterase antagonist is administered intravenously at a dose of between about 5 and 500 μg/kg body weight.
